(19) United States
(12) Patent Application Publication
(10) Pub. No.: US 2014/0066973 A1
(43) Pub. Date: Mar. 6, 2014

(54) Therapy Wrap Device For Applying Pressure To Sciatic-Pain Trigger Points

(71) Applicant: Barbara J. Mahoney, Portland, OR (US)

(72) Inventor: Barbara J. Mahoney, Portland, OR (US)

(21) Appl. No.: 13/975,484

(22) Filed: Aug. 26, 2013

Related U.S. Application Data
(60) Provisional application No. 61/695,414, filed on Aug. 31, 2012.

(51) Int. Cl. A61F 5/30

(52) U.S. Cl. CPC A61F 5/30
USPC 606/201

(57) ABSTRACT

A therapy-wrap device comprises a casing and at least one pressure-applying device. The casing comprise at least one sleeve, and the at least one pressure-applying device is disposed in the at least one sleeve and is adapted to apply pressure to a selected portion of a body of a user when the casing is placed in contacting proximity with the body of the user, such as a muscle trigger point. In one exemplary embodiment, the pressure-applying device is selectably positionable in the at least one sleeve. In use, the pressure that is applied to a muscle trigger point is selectably variable by the user. In one exemplary embodiment, a heat-applying device is disposed in at least one cavity of the casing so that heat can be applied to the selected portion of the body of the user.

### CROSS-REFERENCE TO RELATED APPLICATION

The present patent application claims the benefit of U.S. Provisional Patent Application Ser. No. 61/695,414, filed Aug. 31, 2012, which is incorporated by reference herein.

### BRIEF DESCRIPTION OF THE DRAWINGS

The subject matter disclosed herein is illustrated by way of example and not by limitation in the accompanying figures in which like reference numerals indicate similar elements and in which:

FIGS. 1 and 2 depicts an exemplary embodiment of a therapy-wrap device according to the subject matter disclosed herein; and

FIGS. 3A-3F depict exemplary trigger points that are indicated by an “X” for selected muscles.

### DETAILED DESCRIPTION

As used herein, the word “exemplary” means “serving as an example, instance, or illustration.” Any embodiment described herein as “exemplary” is not to be construed as necessarily preferred or advantageous over other embodiments. Additionally, it will be appreciated that for simplicity and/or clarity of illustration, elements illustrated in the figures have not necessarily been drawn to scale. For example, the dimensions of some of the elements may be exaggerated relative to other elements for illustrative clarity. Further, in some figures only one or two of a plurality of similar elements indicated by reference characters for illustrative clarity of the figure, whereas all of the similar element may not be indicated by reference characters. Further still, it should be understood that although some portions of components and/or elements of the subject matter disclosed herein have been omitted from the figures for illustrative clarity, good engineering, construction and assembly practices are intended.

Any pain felt in the hips and/or legs is generally defined by the public as “sciatica.” The muscles most commonly associated sciatic-type symptoms are the Quadratus Lumborum, the Gluteus Medius, the Gluteus Minimus, the Gluteus Maximus, the Iliopsoas, and the Pirformis. These muscles have trigger points, which at times are irritable portions of the muscles that refer pain to other areas of the body. Direct pressure on a trigger point releases tension in the muscle, thereby allowing a muscle to lengthen and to move with greater flexibility and increased range of motion.

The subject matter disclosed herein provides a therapy-wrap device for applying pressure to sciatic-pain trigger points in muscles that cause sciatic pain. In one exemplary embodiment, the subject matter disclosed herein further comprises a heat-applying device with the pressure-applying device, thereby providing additional comfort and increased blood flow to the affected muscles. In another exemplary embodiment, the subject matter disclosed herein provides an attachment mechanism for comfortably securing and positioning the pressure-applying device in place.

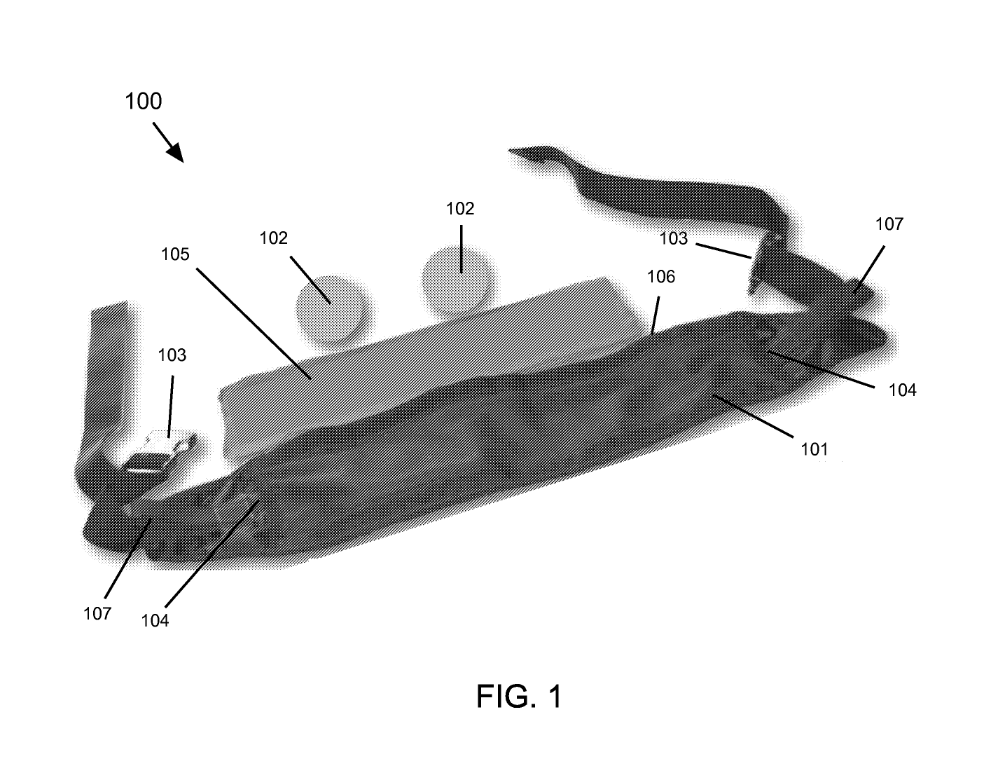

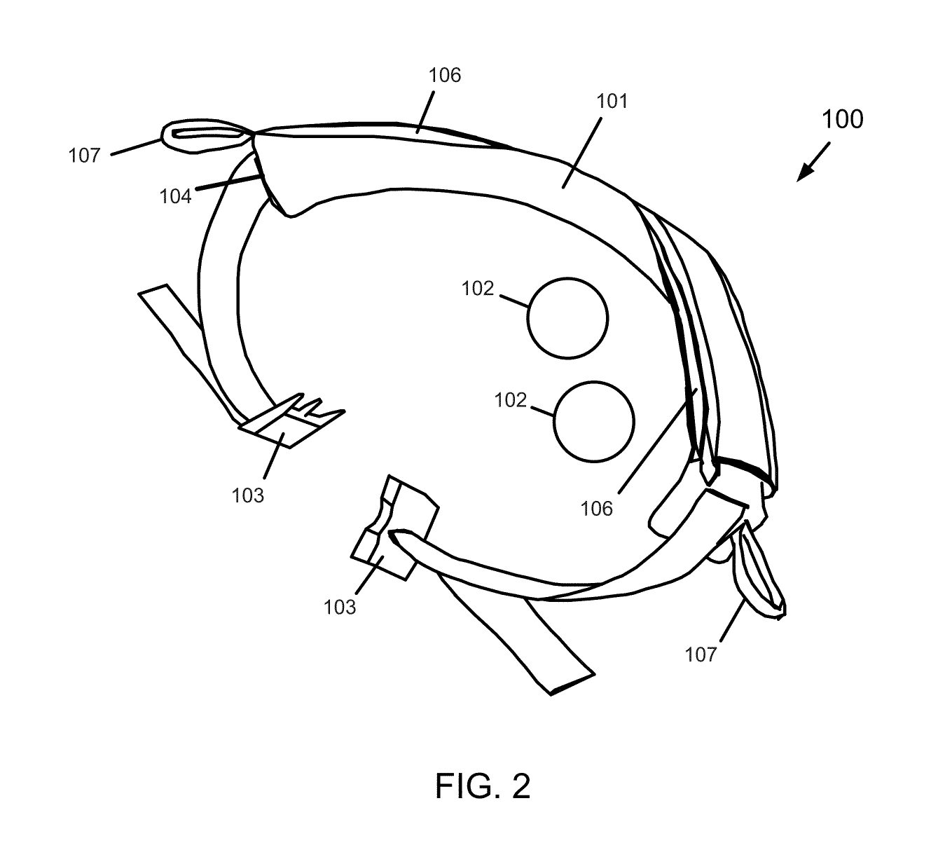

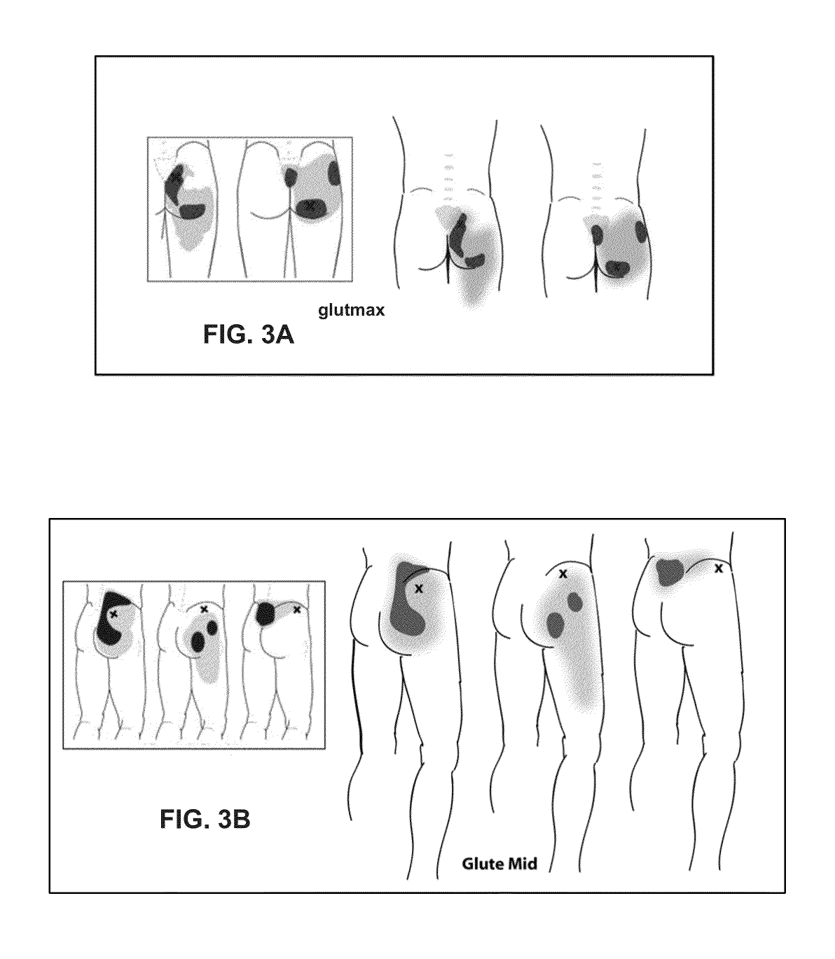

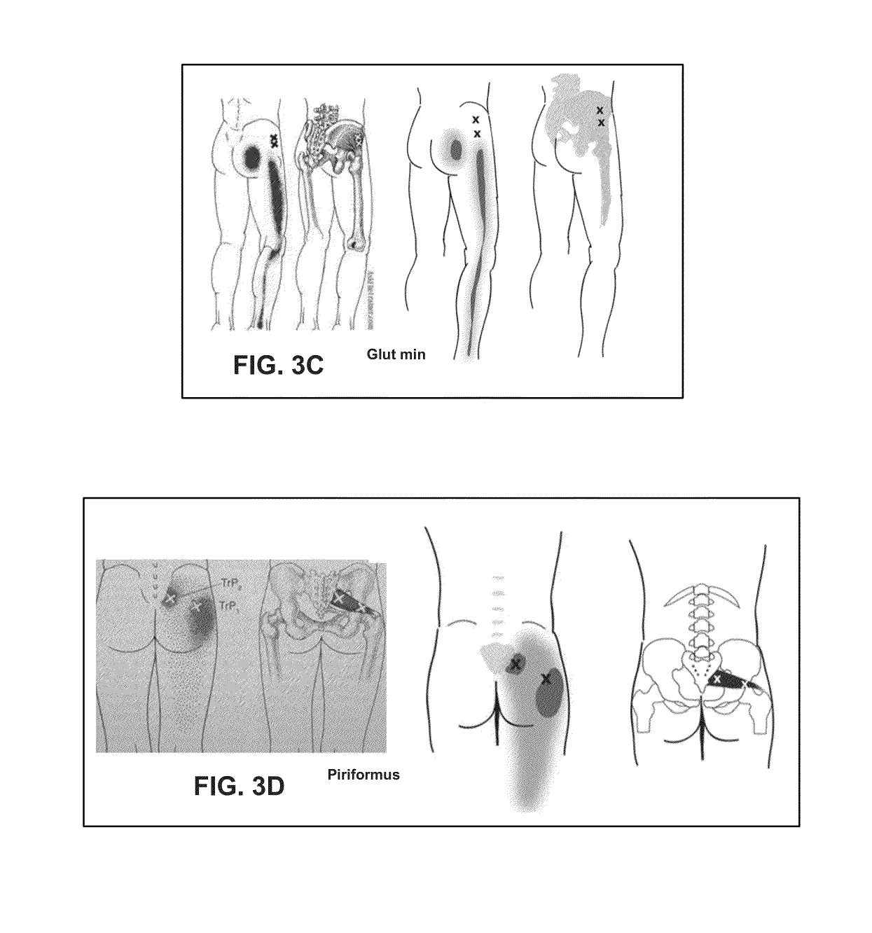

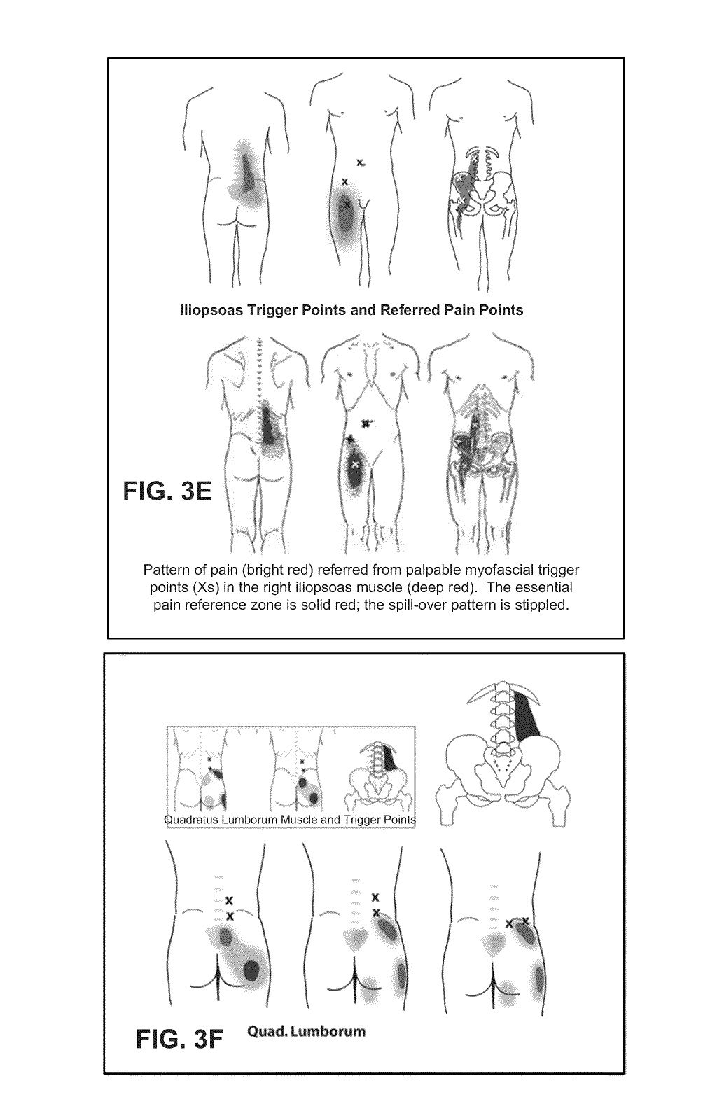

FIGS. 1 and 2 depict an exemplary embodiment of a therapy-wrap device 100 according to the subject matter disclosed herein. Therapy-wrap device 100 comprises a casing 101, at least one pressure-applying device 102, and a fastening device 103. Casing 101 includes a sleeve, or cavity, 104, into which one or more pressure-applying devices 102 are placed. The position of a pressure-applying device 102 within sleeve 104 is selectable by a user. Additionally, the number of pressure-applying devices 102 placed in sleeve 104 is also selectable. In use, one or more pressure-applying devices 102 are placed in sleeve 104 and therapy-wrap device 100 is then positioned around a selected portion of a user's body so that the one or more pressure-applying devices are in proximity to a muscle trigger point, and secured in place by fastening device 103. In one exemplary embodiment, tightening of fastening device 103 allows a user to vary the amount of pressure applied to the muscle trigger point by the one or more pressure-applying devices 102. In another exemplary embodiment, a user vary the amount of pressure applied to the muscle trigger point by applying additional pressure to the one or more pressure-applying devices by, for example, leaning against a stationary surface or object. FIGS. 3A-3F depict exemplary trigger points for selected muscles. The trigger points are indicated by an “X”.

In one exemplary embodiment, fastening device 103 comprises a buckle mechanism that can be selectively tightened by the user to apply a selectably variable amount of pressure to a muscle trigger point. Additionally, fastening device 103 provides stability and comfort for a user. In an alternative exemplary embodiment, fastening device 103 could comprise, but is not limited to, a Velcro-type fastener, a button-type mechanism, or a snap.

In one exemplary embodiment, casing 101 is formed from a fabric-type material selected to provide flexibility and comfort for a user, and that provides structural durability for repeated long-term use. In one exemplary embodiment, casing 101 is formed from several different types of fabric-type materials, such as, but not limited to, cotton, canvas and nylon.

In one exemplary embodiment, pressure-applying devices 102 are spherically shaped and sized to be about the size of a tennis ball, although the claimed subject matter is not so limited. In another exemplary embodiment, pressure-applying devices 102 are hemispherically shaped. In still another exemplary embodiment, a pressure-applying device 103 could be shaped to have a protuberance that is sized and shaped to conveniently apply pressure to a muscle trigger point when disposed in sleeve 104. In one exemplary embodiment, at least one pressure-applying device 102 is formed to be an exterior portion of casing 101.

In one exemplary embodiment, sleeve 104 extends continuously from near one end of therapy-wrap device 100 to near the other end of therapy-wrap device 100. In another exemplary embodiment, therapy-wrap 100 could comprise a plurality of sleeves 104 that allow one or more pressure-applying devices 102 to be selectively placed within one or more of the plurality of sleeves.

In one exemplary embodiment, therapy-wrap device 100 comprises a heat-applying device 105 that can be disposed in a pocket, or cavity, 106 in casing 101. Heat-applying device 105 is capable of applying heat to the selected portion of the body of the user receiving pressure from the pressure-applying devices 102. In one exemplary embodiment, heat-applying device 105 comprises a clay-based device, such as a ThermiPaq-type device available from Thermionics Corporation in Springfield, Ill., that can be heated in a well-known manner in, for example, a microwave oven. In another exemplary embodiment, heat-applying device 105 comprises a battery-powered heater. In still another exemplary embodiment, heat-applying device 105 comprises an electrical heater that is powered from a wall outlet through, for example, a transformer. In yet another exemplary embodiment, heat-applying device 105 comprises flax seeds that can be heated in a well-known manner in, for example, a microwave oven. In still another exemplary embodiment, heat-applying device 105 comprises, but is not limited to, silica beads, micro beads, gel and/or water that can be heated in a well-known manner in, for example, a microwave oven.

In another exemplary embodiment, therapy-wrap device 100 comprises handles, loops or straps 107 that can be gripped by a user to aid in adjusting the positioning of the therapy-wrap device.

Although the foregoing disclosed subject matter has been described in some detail for purposes of clarity of understanding, it will be apparent that certain changes and modifications may be practiced that are within the scope of the appended claims. Accordingly, the present embodiments are to be considered as illustrative and not restrictive, and the subject matter disclosed herein is not to be limited to the details given herein, but may be modified within the scope and equivalents of the appended claims.

What is claimed is:

1. A device, comprising:
a casing comprising at least one sleeve; and
at least one pressure-applying device disposed in the at least one sleeve and adapted to apply pressure to a selected portion of a body of a user when the casing is placed in contacting proximity with the body of the user.

2. The device according to claim 1, wherein the at least one pressure-applying device is selectably positionable in the at least one sleeve.

3. The device according to claim 1, wherein a pressure applied by the at least one pressure-applying device is selectably variable by the user.

4. The device according to claim 1, wherein at least one pressure-applying device is spherically shaped.

5. The device according to claim 1, further comprising a heat-applying device disposed in at least one cavity of the casing, the heat-applying device being capable of applying heat to the selected portion of the body of the user.

6. The device according to claim 5, wherein the at least one pressure-applying device is selectably positionable in the at least one sleeve.

7. The device according to claim 1, further comprising a fastening device coupled to the casing, the fastening device being capable of fastening the casing to the user.

8. The device according to claim 1, wherein the selected portion of the body of the user is a trigger point for a muscle that releases tension associated with the trigger point.

9. The device according to claim 1, further comprising a handle coupled to the casing that is capable of being gripped by a user for selectively positioning the at least one pressure-applying device when the casing is placed in contacting proximity with the body of the user.

10. A device, comprising:
a casing comprising a fastening device and at least one sleeve, the fastening device being capable of fastening the casing to a selected portion of a user's body;
at least one pressure-applying device disposed in the at least one sleeve and adapted to apply pressure to the selected portion of a body of a user when the casing is placed in contacting proximity with the body of the user; and
a heat-applying device disposed in at least one cavity of the casing, the heat-applying device being capable of applying heat to the selected portion of the body of the user.

11. The device according to claim 10, wherein the at least one pressure-applying device is selectably positionable in the at least one sleeve.

12. The device according to claim 11, wherein at least one pressure-applying device is spherically shaped.

13. The device according to claim 10, wherein a pressure applied by the at least one pressure-applying device is selectably variable by the user.

14. The device according to claim 10, wherein the selected portion of the body of the user is a trigger point for a muscle that releases tension associated with the trigger point.

15. The device according to claim 14, wherein the trigger point is associated with sciatic pain of the user.

16. The device according to claim 10, wherein the casing comprises a portion formed from a fabric material.

17. A device, comprising:
a casing comprising a fastening device and at least one sleeve, the fastening device being capable of fastening the casing to a selected portion of a user's body; and
at least one pressure-applying device disposed in the at least one sleeve and adapted to apply pressure to the selected portion of a body of a user when the casing is placed in contacting proximity with the body of the user, the at least one pressure-applying device being selectably positionable in the at least one sleeve.

18. The device according to claim 17, wherein a pressure applied by the at least one pressure-applying device is selectably variable by the user.

19. The device according to claim 18, further comprising a heat-applying device disposed in at least one cavity of the casing, the heat-applying device being capable of applying heat to the selected portion of the body of the user.

20. The device according to claim 17, further comprising a handle coupled to the casing that is capable of being gripped by a user for selectively positioning the at least one pressure-applying device when the casing is placed in contacting proximity with the body of the user.
